(19) United States
(12) Patent Application Publication
(10) Pub. No.: US 2011/0008426 A1
(43) Pub. Date: Jan. 13, 2011

(54) MODIFIED RELEASE PHARMACEUTICAL COMPOSITIONS COMPRISING MYCOPHENOLATE AND PROCESSES THEREOF

(76) Inventors: Rajesh Jain, New Delhi (IN); Sukhjeet Singh, New Delhi (IN)

Correspondence Address: LADAS & PARRY LLP, 26 WEST 61ST STREET, NEW YORK, NY, 10023, US

(21) Appl. No.: 12/920,512

(22) PCT Filed: Mar. 4, 2009
(86) PCT No.: PCT/IN09/00148

§ 371 Date: Sep. 1, 2010

(30) Foreign Application Priority Data
Mar. 5, 2008 (IN) 549/DEL/2008

(51) Int. Cl. A61K 9/52; A61K 9/22; A61K 31/365; A61K 9/14; A61K 9/26; A61K 9/24; A61P 37/06; A61P 37/02

(52) U.S. Cl. 424/457; 424/468; 514/470; 424/490; 424/469; 424/465; 424/472

(57) ABSTRACT

Modified release pharmaceutical compositions comprising mycophenolate as the active agent or its pharmaceutically acceptable salts, esters, polymorphs, isomers, prodrugs, solvates, hydrates, or derivatives thereof, wherein the said composition exhibits a biphasic release profile when subjected to in-vitro dissolution and/or upon administration in-vivo are provided. The composition provides a drug release in a manner such that the drug levels are maintained above the therapeutically effective concentration (EC) constantly for an extended duration of time. Further, the difference between the maximum plasma concentration of the drug (Cmax) and the minimum plasma concentration of the drug (Cmjn), and in turn the flux defined as ((Cmax−Cmjn)/Cavg) is minimal. The present invention also provides process of preparing such dosage form compositions and prophylactic and/or therapeutic methods of using such compositions.

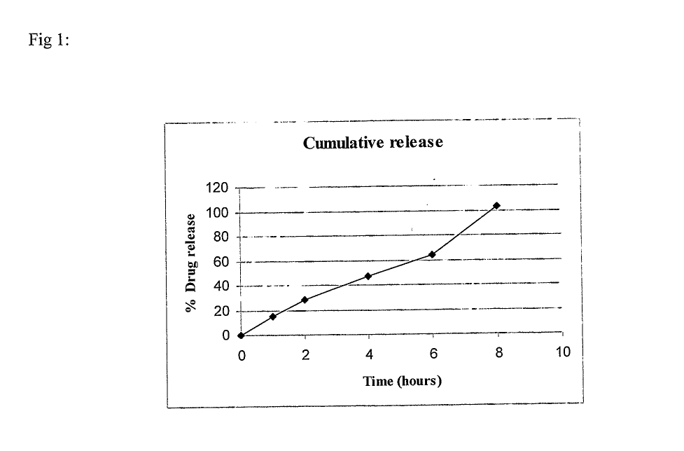

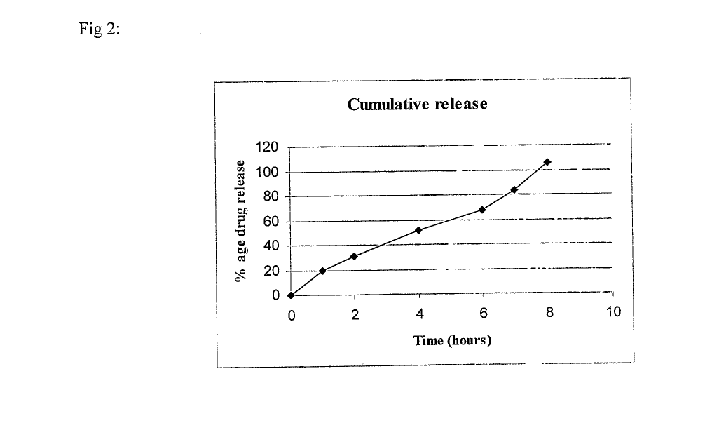

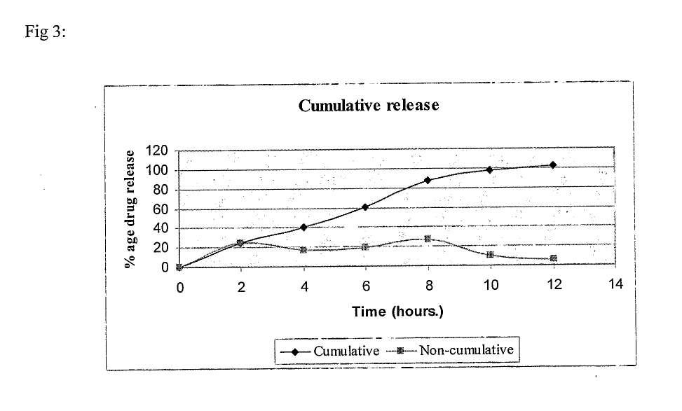

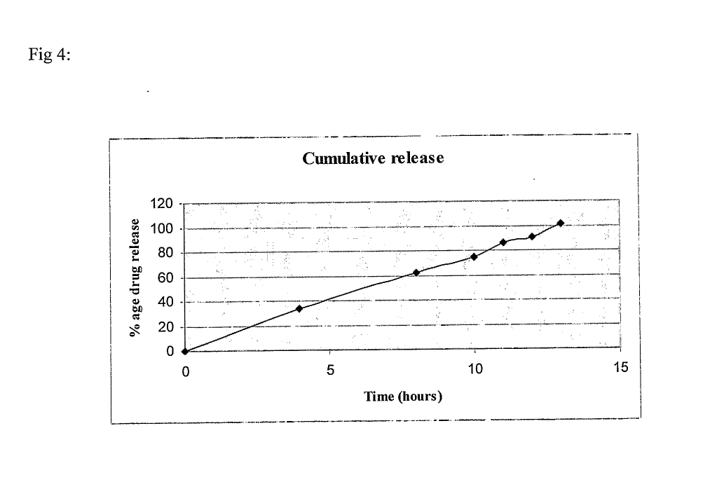

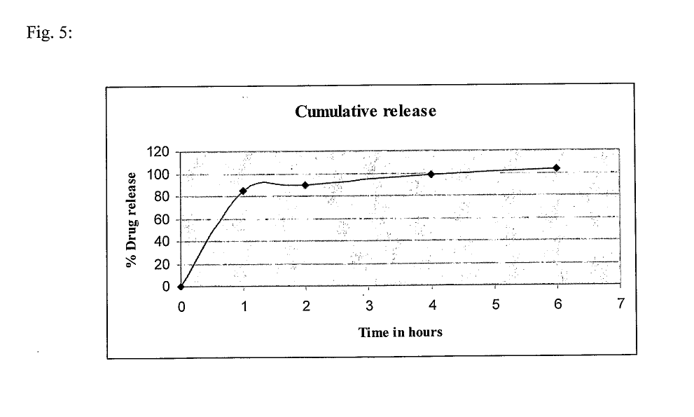

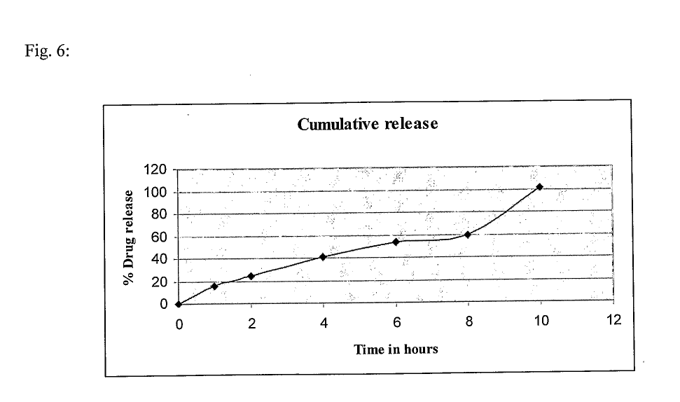

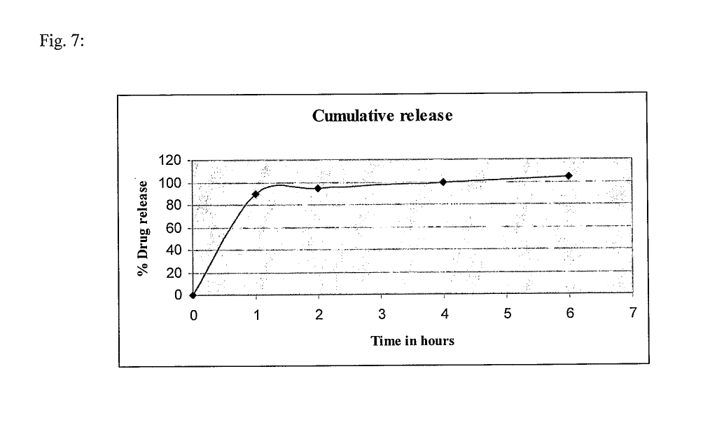

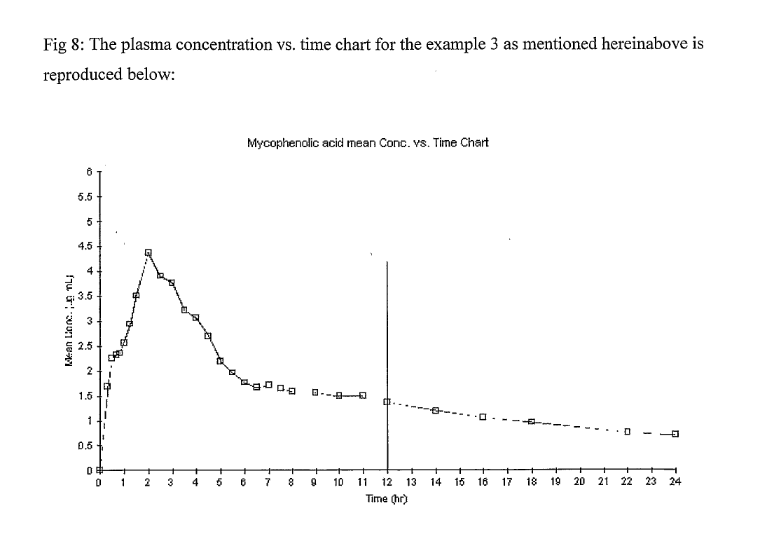

### FIELD OF THE INVENTION

The present invention relates to modified release pharmaceutical compositions comprising mycophenolate as the active agent or its pharmaceutically acceptable salts, esters, polymorphs, isomers, prodrugs, solvates, hydrates, or derivatives thereof, wherein the said composition exhibits a biphasic release profile when subjected to in-vitro dissolution and/or upon administration in-vivo. The composition essentially comprises an immediate release fraction (IR) and at least one extended release fraction and provides a drug release in a manner such that the drug levels are maintained above the therapeutically effective concentration (EC) but below the toxic concentration (TC) constantly for an extended duration of time. Further, the difference between the maximum plasma concentration of the drug (Cₘₐₓ) and the minimum plasma concentration of the drug (Cₘᵢₙ), and in turn the flux defined as ((Cₘₐₓ−Cₘᵢₙ)/C_{avg}) is relatively less as compared to the approved compositions of mycophenolate such as Myfortic® (mycophenolate sodium). Furthermore, the compositions of the present invention are designed to release mycophenolate in-vivo in a manner so that it substantially alleviates or at least reduces the chances of causing any associated gastrointestinal side effect(s) without compromising the bioavailability/efficacy of the said active agent. The present invention also provides process of preparing such dosage form compositions and prophylactic and/or therapeutic methods of using such compositions. The compositions of the present invention are safe, effective and well-tolerated, and are useful for the management such as prophylaxis, amelioration and/or treatment of immunosuppressant indicated disease(s)/disorder(s) especially for the treatment or prevention of organ, tissue or cellular allograft or xenograft rejection, e.g. after transplant, or the management of immune-mediated diseases (autoimmune diseases).

### BACKGROUND OF THE INVENTION

Mycophenolic acid (MPA) was first isolated in 1896, and has been extensively investigated as a pharmaceutical of potential commercial interest. It is known to have anti-tumor, anti-viral, immunosuppressive, anti-psoriatic, and anti-inflammatory activity [see e.g. W. A. Lee et al, Pharmaceutical Research (1990), 7, p. 161-166 and references cited therein]. A derivative of MPA such as mycophenolate mofetil (MMF) had been introduced commercially in the US and elsewhere under the brand name CellCept® as an immunosuppressant in the immediate release form for the treatment or prevention of organ or tissue transplant rejection. A pharmaceutical delayed release composition comprising mycophenolate sodium (Myfortic®) has been approved for marketing in the United States. It has been concluded in a study that mycophenolate sodium is therapeutically equivalent to MMF at equimolar MPA doses. 769.4 mg of mycophenolate sodium contains equimolar amounts of MPA compared with 1000 mg of MMF [Progress in Transplantation; June 2004; Gabardi, S et al]. Mycophenolate Sodium salts are known, e.g. in South African Patent 68/4959. U.S. Pat. Nos. 6,025,391, 6,172,107 and 6,306,900 describe pharmaceutical compositions comprising a mycophenolate salt, wherein the composition is enteric coated thereby preventing the release of the mycophenolate salt in the stomach, and releasing the mycophenolate salt in the upper part of the intestinal tract. However the major limitation of formulating such a composition of mycophenolate is that although the enteric coat is intended to prevent release of the drug in the stomach to prevent associated side effects, the clinical study results in 423 de novo kidney allograft recipients indicates that the incidence of GI adverse events was 79.8% with mycophenolate sodium and 77.1% with MMF (P═NS) and also the frequency of dosage reductions, discontinuation, or temporary interruptions of therapy secondary to GI toxicities were comparable.

TABLE 1
Adverse Events (%) in Controlled de novo and Maintenance
Renal Studies Reported in 20% of Patients

| | | |
|---|---|---|
| Side effects | MMF (%) | MPS (%) |

Blood and Lymphatic System disorder

| | | |
|---|---|---|
| Anemia | 21.6 | 21.9 |
| Leukopenia | 19.2 | 20.5 |

Gastrointestinal System Disorders

| | | |
|---|---|---|
| Constipation | 38.0 | 39.5 |
| Nausea | 29.1 | 27.1 |
| Diarrhoea | 24.8 | 23.5 |
| Vomiting | 20 | 23 |
| Dyspepsia | 22.5 | 19 |

Infections and infestations

| | | |
|---|---|---|
| Urinary Tract Infection | 29.1 | 33.3 |
| CMV infections | 20.2 | 18.1 |

Nervous System Disorders

| | | |
|---|---|---|
| Insomnia | 23.5 | 23.8 |

The etiology of the observed gastrointestinal effects due to MMF was reviewed by Behrend et al (Adverse Gastrointestinal Effects of Mycophenolate Mofetil Aetiology, Incidence and Management, 2001). It was concluded by the authors that the gastrointestinal adverse effects of MMF were mainly related to Cₘₐₓ, of the active moiety i.e., MPA whereas the efficacy was related to AUC. A few other authors have also reported that there is a correlation between plasma concentration of MPA (C30) and side effects (Correlation of mycophenolic acid pharmacokinetic parameters with side effects in kidney transplant patients treated with mycophenolate mofetil, Clinical Chemistry, 2001, Mourad et al).

The insignificant difference in the adverse effects of MMF and EC-MPS (Table 1) can be attributed to high Cₘₐₓ value of MPA in both the cases. The values of Cₘₐₓ for MMF (1 g b.i.d) and EC-MPS (720 mg b.i.d) were found to be 21.3 and 18.93 μg/mL respectively (Am J Transplant, 2007, Budde et al.).

Hence, there still exists an unmet need for developing a suitable dosage form which releases the drug mycophenolate throughout the GIT in a modified release manner to achieve the desired bioavailability with a lesser difference in the peak (Cₘₐₓ) and trough (Cₘᵢₙ) drug concentrations, a lesser flux and providing a therapeutic concentration of the drug for an extended duration substantially devoid of associated side effects for achieving better patient compliance. Further there is a need to develop compositions which can provide a biphasic/multiphasic release of mycophenolate such that the Area-under-the-drug plasma concentration-time-curve upto 12 hours (AUC₀₋₁₂) is as close as possible to the Area-under-the-drug plasma concentration-time-curve from 12-24 hours (AUC₁₂₋₂₄) in order to ensure that the therapeutic concentrations of the active agent are maintained for a longer duration of time. The present invention provides such compositions of mycophenolate. PCT Publication No. WO2006024479 discloses a composition comprising mycophenolic acid, a salt or a prodrug thereof in a modified release form. Such compositions are intended to improve the drug distribution in the intestine, and to delay the delivery of the drug substance to the intestinal tract. Multiparticulate systems such as granules, pellets, beads and minitablets are disclosed. Also disclosed are coated pellets and granules compressed into rapid disintegrating tablets. However no single unit dosage forms either uncoated or coated for providing a sustained release of mycophenolate sodium particularly throughout the GIT are disclosed. Further the multiparticulate system of the said publication if formulated as a compressed dosage form such as tablet, the application of compression force in a compression machine will lead to rupturing of the coated multiple units resulting in loss of uniformity of the coating layer over the entire unit (pellet or granule) thus producing variable and unpredictable release of the active agent from such compressed forms. Furthermore the multiparticulate system of the present invention lacks patient compliance since the multiple units such as pellets or granules if administered orally to a patient will cause an unpleasant and gritty feeling in the mouth and would be difficult to swallow since it might stick to the oral cavity. Still further, such multiparticulate systems would require the use of an additional taste-masking agent in the composition.

PCT Publication No. WO2005034916 describes a composition comprising mycophenolic acid, a salt or a prodrug thereof in multiparticulate form such that the compositions disintegrate or dissolve in the mouth, stomach or small intestine to give multiparticles, wherein the multiparticles are enteric coated. Such compositions do not provide a uniform sustained release of the active agent throughout the GIT; instead it releases the drug only in the intestine. Further the drug release from the multiparticulate systems are generally non-uniform since it is extremely difficult to predetermine and/or control the behavior of such systems upon in vivo administration.

Oral drug delivery systems are exposed to a wide range of highly variable conditions inside the GI tract such as pH, agitation intensity, gastric emptying time and composition of the gastrointestinal fluids during its transit through the digestive tract. Thus the formulation of suitable modified release drug delivery systems should take into consideration the physico-chemical and physiological environment of the gastrointestinal tract as well besides the formulation aspects. The conventional oral approaches to controlled or sustained or prolonged release formulation known in the art are not applicable to a variety of drugs having an “absorption window” in the stomach or upper parts of small intestine. Furthermore, it is advantageous to make a modified release dosage form which releases the drug in a biphasic manner thereby achieving a better therapeutic efficacy.

A review of the prior art literature shows that no formulation has been approved till date for a modified release pharmaceutical composition comprising mycophenolate as the active agent or its pharmaceutically acceptable salts, esters, polymorphs, isomers, prodrugs, solvates, hydrates, or derivatives thereof. Mycophenolate formulations disclosed in prior art are particularly multiparticulate and/or enteric-coated (delayed release) systems which suffer from several disadvantages as discussed earlier. Hence there still exists a need for commercially acceptable preferably single unit dosage forms for oral administration with good patient convenience and acceptance. Also there is still an unmet need to develop especially once-a-day modified release mycophenolate compositions comprising substantially high dose of said active agent with a fraction of the drug meant for immediate release for a rapid onset of action and a fraction of the drug meant for extended release for prolonged duration of action. Also such compositions should be substantially devoid of inter- and intra-patient variability. Particularly there still exists a need for developing oral modified release mycophenolate compositions which are stable and easily swallowable upon oral administration, possess appreciable bioavailability characteristics, are well-tolerated and safe, and exhibit a drug release for a therapeutic effect. Also there exists a need to develop oral modified release pharmaceutical compositions comprising mycophenolate for prophylactic and/or therapeutic use, which can release the drug in a manner such that the drug levels are maintained above the therapeutically effective concentration (EC) constantly for an extended duration of time. Further, the difference between the maximum plasma concentration of the drug (Cₘₐₓ) and the minimum plasma concentration of the drug (Cₘᵢₙ), and in turn the flux as herein defined ((Cₘₐₓ−Cₘᵢₙ)/C_{avg}) should be relatively less and thus should provide a flattened drug release profile for an extended time period wherein the Area-under-curve (AUC) practically remains unchanged as compared to the approved compositions of mycophenolate such as Myfortic® (mycophenolate sodium). Furthermore, the compositions of the present invention are designed to release mycophenolate in-vivo in a manner so that it substantially alleviates or at least reduces the chances of causing any associated gastrointestinal side effect(s) without compromising the bioavailability of drug.

Mycophenolate mofetil (MMF) is indicated for the prophylaxis of organ rejection in patients receiving allogeneic renal transplants and in patients receiving allogeneic cardiac transplants. It is used concomitantly with cyclosporine and corticosteroids. However, despite its efficacy, immunosuppressive therapy with MMF has been limited by its tolerability. An enteric-coated formulation of Mycophenolate sodium (EC-MPS) has similar efficacy and safety compared to MMF. Instead of rapid release of Mycophenolic acid (MPA) into the stomach from the prodrug MMF, the enteric coating allows delayed release of MPA into the small intestine. No significant difference in the adverse effects of MPS (blood and lymphatic system disorders 41.8%, constipation 38%, nausea 29%, diarrhea 23.5%, vomiting 23%, urinary tract infections 29.1%, CMV infection 20.2%) has been reported when compared to MMF (blood and lymphatic system disorders 42.4%, constipation 39.5%, nausea 27.1%, diarrhea 24.8%, vomiting 20%, urinary tract infections 33.3%, CMV infection 18.1%) [www.rxlist.com]. The usual recommended dose of mycophenolate is 1 to 3 gm per day. Using the presently marketed immediate release tablet compositions containing 180 and 360 mg of Mycophenolic acid which is active moiety, a patient receiving a 3.0 gram daily dose is required to take about six tablets each day, giving rise to patient inconvenience and noncompliance. According to the recommended dose, at least two tablets needs to be administered twice daily which lead to patient compliance concerns. Hence, a modified release/controlled release composition of Mycophenolate shall definitely increase the patient compliance.

Although the adverse effect (AE) profile of mycophenolate is comparatively benign, gastrointestinal AE are a major concern. These AE which include diarrhea, abdominal pain, nausea, vomiting, gastrointestinal infection etc (Begrend, 2001) are partially explained by the increased immune suppression, by the mode of action and by interactions particularly with other immunosuppressants. The high local concentrations of MPA, not reflecting systemic exposure may contribute to the gastrointestinal adverse effects (Van Gelder et al., 1999). The cyclosporine may also contribute to a locally high concentration of MPA (Begrend, 2001). Additionally, enterohepatic recirculation may lead to high MPA concentrations. In several studies withdrawal of mycophenolate because of gastrointestinal AE was significantly related to the mycophenolate dose. Therefore, all adverse events seem to be related to the peak plasma concentration of the drug (Cₘₐₓ), whereas immunosuppressant activity is related to total exposure i.e. Area-under-the-drug plasma concentration-time-curve (AUC). This relationship offers opportunities for the management of gastrointestinal adverse effects (Mourad et al., 2001). Takahashi et al. (1995) concluded that patients with low AUC for MPA appear to be at high risk for experiencing graft rejection whereas high target concentration can increase toxicity. Also maintenance of trough plasma concentration of the drug (Cₘᵢₙ) levels (approx 2 μg/mL) for longer duration is necessary to prevent rejection (Yau et al., 2007, Wollenberg et al., 1998; Krumme et al., 1998).

Steady-state MPA concentrations are predictive of the risk for acute rejection in transplant patients. The fact that increasing dose-interval MPA AUC values lowers the risk for acute rejection is based on retrospective reviews of clinical studies in renal and heart transplant patients. A review of this study data concluded that maintenance of transplant patients within a target range of 30 to 60 mg*hr/mL, assuming concomitant therapy was cyclosporine and corticosteroids, would assure a risk of acute rejection of less that 10% (Hale et al., 1998). An analysis of MPA pharmacokinetic data developed in a multicenter investigation in pediatric renal transplant patients also concluded that target range of 30 to 60 mg*hr/mL provides for significant reduction in acute rejection and would assure avoidance of chronic exposure to unnecessarily high MPA concentrations. A mycophenolic acid AUC of 15 μg h/mL would be expected to yield 50% of maximal achievable efficacy, whereas 25 μg h/mL and 40 μg h/mL would yield 90% efficacy, respectively (Hale et al., 1998).

Keeping in view all the abovementioned facts of the drug/active agent mycophenolate, it appears that simply making compositions having a sustained or controlled behavior is not beneficial; instead a biphasic/multiphasic release of drug from a modified release composition would be the best approach to develop an effective, safe and tolerable dosage form of mycophenolate. There is a need to develop a modified release dosage form which will have AUC and Cₘᵢₙ as nearly close to immediate release (given multiple times a day) but having Cₘₐₓ lesser than immediate release form. It therefore appears that a modified release tablet composition that produces a biphasic/multiphasic profile may be the best approach for a comparable once-daily or twice-daily product. Such a product will be able to maintain minimum effective levels for a longer period of time and will ensure lower rejection by the body. Additionally, it will be devoid of adverse effects.

Hence there is need for the formulations which can achieve the desired AUC and Cₘᵢₙ values (about 2 μg/mL). At the same time, the formulation should have low Cₘₐₓ so that the adverse effects are low. This can be achieved using controlled release formulation with biphasic release profile. The compositions according to the present invention addresses the problems described herein before in the prior arts and thus provide a significant advancement in the said art.

### SUMMARY OF THE INVENTION

It is an objective of the present invention to provide modified release pharmaceutical compositions comprising mycophenolate as the active agent or its pharmaceutically acceptable salts, esters, polymorphs, isomers, prodrugs, solvates, hydrates, or derivatives thereof, at least one release controlling material(s) and optionally one or more pharmaceutically acceptable excipient(s), wherein the said composition provides a biphasic release profile of the active agent when subjected to in-vitro dissolution and/or upon administration in-vivo.

It is an objective of the present invention to provide modified release pharmaceutical compositions comprising mycophenolate as the active agent or its pharmaceutically acceptable salts, esters, polymorphs, isomers, prodrugs, solvates, hydrates, or derivatives thereof, which provides a biphasic release of the active agent such that the drug levels are maintained above the therapeutically effective concentration (EC) constantly for an extended duration of time and also one or more gastro-intestinal adverse effects associated with therapy using mycophenolate is substantially reduced or alleviated.

It is a further objective of the present invention to provide oral modified release pharmaceutical compositions comprising mycophenolate as the active agent or its pharmaceutically acceptable salts, esters, polymorphs, isomers, prodrugs, solvates, hydrates, or derivatives thereof, wherein the said compositions provide a biphasic/multiphasic release of mycophenolate such that the Area-under-the-drug plasma concentration-time-curve upto 12 hours (AUC₀₋₁₂) is as close as possible to the Area-under-the-drug plasma concentration-time-curve from 12-24 hours (AUC₁₂₋₂₄) thereby, ensuring that the therapeutic concentrations of the active agent are maintained for a longer duration of time.

Further, it is an objective of the present invention to provide modified release pharmaceutical compositions comprising mycophenolate as the active agent or its pharmaceutically acceptable salts, esters, polymorphs, isomers, prodrugs, solvates, hydrates, or derivatives thereof, wherein the ratio of AUC₀₋₁₂ to AUC₁₂₋₂₄ is from about 4:1 to about 1:1 thereby ensuring that the therapeutic concentrations of the active agent are maintained for a longer duration of time.

It is another objective of the present invention to provide modified release pharmaceutical compositions comprising mycophenolate as the active agent or its pharmaceutically acceptable salts, esters, polymorphs, isomers, prodrugs, solvates, hydrates, or derivatives thereof, wherein the ratio of AUC₀₋₁₂ to AUC₁₂₋₂₄ is from about 3:1 to about 1:1 thereby ensuring that the therapeutic concentrations of the active agent are maintained for a longer duration of time.

It is a preferred objective of the present invention to provide modified release pharmaceutical compositions comprising mycophenolate as the active agent or its pharmaceutically acceptable salts, esters, polymorphs, isomers, prodrugs, solvates, hydrates, or derivatives thereof, wherein the ratio of AUC₀₋₁₂ to AUC₁₂₋₂₄ is from about 2.5:1 to about 1:1 thereby ensuring that the therapeutic concentrations of the active agent are maintained for a longer duration of time.

It is also an objective of the present invention to provide modified release pharmaceutical compositions comprising mycophenolate as the active agent or its pharmaceutically acceptable salts, esters, polymorphs, isomers, prodrugs, solvates, hydrates, or derivatives thereof, which can release the drug in a manner such that the difference between the maximum plasma concentration of the drug (Cₘₐₓ) and the minimum plasma concentration of the drug (Cₘᵢₙ), and in turn the flux as herein defined ((Cₘₐₓ−Cₘᵢₙ)/C_{avg}) is relatively less and provides a flattened drug release profile for an extended time period wherein the Area-under-curve (AUC) practically remains unchanged for a substantially longer period of time.

It is another objective of the present invention to provide modified release pharmaceutical compositions comprising mycophenolate as the active agent or its pharmaceutically acceptable salts, esters, polymorphs, isomers, prodrugs, solvates, hydrates, or derivatives thereof, wherein the drug is released in-vivo for an extended period of time in a manner so that it substantially alleviates or at least reduces the chances of causing any associated gastrointestinal side effect(s) without compromising the bioavailability of the drug.

It is an objective of the present invention to provide modified release pharmaceutical compositions comprising mycophenolate as the active agent or its pharmaceutically acceptable salts, esters, polymorphs, isomers, prodrugs, solvates, hydrates, or derivatives thereof, at least one release controlling material(s) and optionally one or more pharmaceutically acceptable excipient(s), wherein the said composition exhibits a biphasic profile of the active agent when subjected to in-vitro dissolution and/or upon administration in-vivo, and wherein the said biphasic release is pH independent i.e. the composition is designed in such a manner that it will not restrict the drug release with respect to various GIT pH.

It is another objective of the present invention to provide modified release pharmaceutical compositions comprising mycophenolate or its pharmaceutically acceptable salts, esters, polymorphs, isomers, prodrugs, solvates, hydrates, or derivatives thereof as the active agent such that the said active agent is adapted to release over a predetermined time period exhibiting a biphasic release profile, wherein the first phase is an immediate release phase and the second phase is an extended release phase or vice versa.

It is another objective of the present invention to provide process of preparing the modified release pharmaceutical compositions according to the present invention comprising mycophenolate or its pharmaceutically acceptable salts, esters, polymorphs, isomers, prodrugs, solvates, hydrates, or derivatives thereof as the active agent.

It is yet another objective to provide method of using the composition of the present invention for the management such as prophylaxis, amelioration and/or treatment of immunosuppressant indicated disease(s)/disorder(s) especially for the treatment and/or prevention of organ, tissue or cellular allograft or xenograft rejection, e.g. after transplant, or the management of immune-mediated diseases (autoimmune diseases), which comprises administering to a subject in need thereof the composition comprising a pharmaceutically effective amount of mycophenolate or its pharmaceutically acceptable salts, esters, polymorphs, isomers, prodrugs, solvates, hydrates, or derivatives thereof as the active agent.

It is yet another objective of the present invention to provide use of the pharmaceutical composition comprising pharmaceutically effective amount of mycophenolate or its pharmaceutically acceptable salts, esters, polymorphs, isomers, prodrugs, solvates, hydrates, or derivatives thereof in the preparation of a medicament for the management such as prophylaxis, amelioration and/or treatment of immunosuppressant indicated disease(s)/disorder(s) especially for organ, tissue or cellular allograft or xenograft rejection, such as after transplant, or the management of immune-mediated diseases (autoimmune diseases).

The pharmaceutical compositions of the present invention are intended for once-a-day or twice-a-day administration, preferably for once-a-day administration. The composition releases the active agent mycophenolate sodium in a desired manner so as to maintain prophylactic and/or therapeutic levels of the active agent in the plasma for an extended period of time devoid of any substantial drug related toxicity, and also can be prepared in an easy and cost-effective manner.

### DETAILED DESCRIPTION OF THE INVENTION

Developing a modified release dosage form composition particularly for drugs like Mycophenolate sodium having some peculiar properties such as high dose requirement, high water solubility and insolubility in gastric (acidic) pHs, predominant absorption in small intestine and re-absorption through entero-hepatic circulation is a challenging task. However, a modified release dosage form for Mycophenolate is expected to provide better or improved patient compliance than the existing conventional dosage forms. As per the recommended dosage regimen of mycophenolate, a patient needs to take four tablets in a day and sometimes it increases up to 6 tablets in a day depending on the condition. So developing MR formulation as once daily dosage form with acceptable tablet properties such as weight, appearance etc. is highly desirable and will definitely improve the patient compliance.

Particularly the gastro-intestinal (GI) adverse effects of mycophenolate are a major concern associated with the therapy with the said drug. These effects are partially explained by the increased immune suppression, by the mode of action and by interactions particularly with other immunosuppressants. The data suggest that the GI adverse effects are mainly related to Cₘₐₓ, whereas the efficacy is related to AUC. The GI adverse effects such as abdominal pain/discomfort, constipation, nausea, diarrhea, vomiting and the like are particularly due to high levels of the active agent at a given point of time. At the same time, the levels of the active agent come down quickly and fall below the therapeutic concentrations, thus requiring another dose of the drug. This is particularly the case when only immediate-release conventional dosage forms are used for therapy. In other case wherein a sustained release composition containing mycophenolate is administered, though the incidence of GI side effects may not be so high as in the case of an immediate release dosage form, but such compositions are not able to sustain the release of the active agent for extended duration since the half-life of the drug is short and the plasma levels fall below the therapeutically effective concentrations quickly particularly after 4-6 hours after administration, more particularly after 12 hours of administration. The compositions of the present invention alleviates such drawbacks by providing a biphasic release of the active agent wherein one part of the active agent is made as a fast release fraction and the other part of the active agent is made as a sustained release fraction such that not only a therapeutically effective concentration of the active agent is maintained for an extended duration, but also the associated GI adverse effects are substantially minimized. Particularly the compositions of the present invention can be therefore administered once-a-day.

Further, the development of a modified release dosage form for mycophenolate sodium is challenging due to the pH-dependent solubility of the drug i.e. high solubility of the drug in pH above 6.0 and poor solubility in pH below 6.0. Furthermore, it is challenging to develop modified release dosage forms of mycophenolate sodium which are robust, and whose dissolution behavior does not depend on the state of digestion or dosage form transit through the gastrointestinal tract. The inventors of the present invention have been able to develop modified release compositions comprising mycophenolate sodium thus demonstrating a significant advancement over the prior art. The present invention provides a stable modified release single unit dosage form composition such as matrix tablets which could provide substantially linear drug release for a prolonged duration of time during the transit through varying pH of the GIT.

Generally controlled release pharmaceutical formulations which provide zero order release characteristics have been considered most desirable because such a mechanism would theoretically provide constant drug levels. This is based on the assumption that the rate of elimination is determinative of the release rate of the medicament from the formulation, rather than the rate of absorption, and the like. However, for drugs such as mycophenolate which are not very soluble in the stomach and where the absorption is desired for a prolonged period of time, a biphasic or multiphasic release mechanism is considered to be desirable, which the present invention provides. Multiphasic release according to the present invention is defined as the release of the active agent in at least two phases. For example, the multiphasic release can be a biphasic one characterized by an initial high rate followed by a slower rate as the dosage form passes the upper portion of the small intestine where absorption is maximum and finally another higher rate as the dosage form passes into the further end of the intestine where absorption is less than before. Biphasic or multiphasic release for compositions comprising mycophenolate is considered to be advantageous because it allows to compensate for changing absorption rates of the drug in the gastrointestinal tract by providing a rapid onset of action (for e.g. when the formulation is located in the stomach and small intestine) and compensate for relatively slow absorption by providing a relatively rapid release rate (for e.g. when the formulation is located in the large intestine).

The inventors of the present invention have done extensive research and experimentation to design single unit modified release dosage form composition comprising mycophenolate particularly mycophenolate sodium which provides a biphasic or multiphasic release of the drug.

The present invention provides modified release pharmaceutical compositions comprising mycophenolate as the active agent or its pharmaceutically acceptable salts, esters, polymorphs, isomers, prodrugs, solvates, hydrates, or derivatives thereof, at least one release controlling material(s) and optionally one or more pharmaceutically acceptable excipient(s), wherein the said composition exhibits a multiphasic release profile when subjected to in-vitro dissolution and/or upon administration in-vivo.

In an embodiment, the compositions of the present invention release the drug in such a manner so that the drug levels are maintained above the therapeutically effective concentration (EC) constantly for an extended duration of time. In another embodiment, the difference between the maximum plasma concentration of the drug (Cₘₐₓ) and the minimum plasma concentration of the drug (Cₘᵢₙ), and in turn the flux is relatively less as compared to the approved compositions of mycophenolate such as Myfortic® (mycophenolate sodium). In a further embodiment, the composition of the present invention provides a flattened drug release profile for an extended time period wherein the Area-under-curve (AUC) practically remains unchanged for a substantially longer period of time.

In context of the present invention; “flux of the drug release” is defined as the difference between the maximum (Cₘₐₓ) and minimum (Cᵣₐₘ) therapeutic concentrations of the drug divided by the average concentration (C_{avg}) of the drug over the period of about 24 hours.

$$Flux = \frac{{C}_{max} - {C}_{min}}{{C}_{avg}}$$

In an embodiment, the present invention provides modified release pharmaceutical compositions comprising mycophenolate as the active agent or its pharmaceutically acceptable salts, esters, polymorphs, isomers, prodrugs, solvates, hydrates, or derivatives thereof, wherein the drug is released in-vivo for an extended period of time in a manner so that it substantially alleviates or at least reduces the chances of causing any associated gastrointestinal side effect(s) without compromising the bioavailability of the drug. The oral modified release mycophenolate compositions which are stable and easily swallowable upon oral administration, possess appreciable bioavailability characteristics, are well-tolerated and safe, and exhibit a drug release for a therapeutic effect.

In a preferred embodiment of the present invention, the modified release pharmaceutical compositions comprising mycophenolate as the active agent or its pharmaceutically acceptable salts, esters, polymorphs, isomers, prodrugs, solvates, hydrates, or derivatives thereof, provide a biphasic/multiphasic release of mycophenolate such that the Area-under-the-drug plasma concentration-time-curve upto 12 hours (AUC₀₋₁₂) is relatively close to the Area-under-the-drug plasma concentration-time-curve from 12-24 hours (AUC₁₂₋₂₄) thereby ensuring that the therapeutic concentrations of the active agent are maintained for a longer duration of time.

In a embodiment of the present invention, the modified release pharmaceutical compositions comprising mycophenolate as the active agent or its pharmaceutically acceptable salts, esters, polymorphs, isomers, prodrugs, solvates, hydrates, or derivatives thereof, wherein the ratio of AUC₀₋₁₂ to AUC₁₂₋₂₄ is from about 4:1 to about 1:1 thereby ensuring that the therapeutic concentrations of the active agent are maintained for a longer duration of time.

In another embodiment of the present invention, the modified release pharmaceutical compositions comprising mycophenolate as the active agent or its pharmaceutically acceptable salts, esters, polymorphs, isomers, prodrugs, solvates, hydrates, or derivatives thereof, wherein the ratio of AUC₀₋₁₂ to AUC₁₂₋₂₄ is from about 3:1 to about 1:1 thereby ensuring that the therapeutic concentrations of the active agent are maintained for a longer duration of time.

In a preferred embodiment of the present invention, the modified release pharmaceutical compositions comprising mycophenolate as the active agent or its pharmaceutically acceptable salts, esters, polymorphs, isomers, prodrugs, solvates, hydrates, or derivatives thereof, wherein the ratio of AUC₀₋₁₂ to AUC₁₂₋₂₄ is from about 2.5:1 to about 1:1 thereby ensuring that the therapeutic concentrations of the active agent are maintained for a longer duration of time.

In an embodiment, the present invention provides modified release pharmaceutical compositions comprising mycophenolate or its pharmaceutically acceptable salts, esters, polymorphs, isomers, prodrugs, solvates, hydrates, or derivatives thereof as the active agent such that the said active agent is adapted to release over a predetermined time period exhibiting a biphasic release profile, wherein the first phase is an immediate release phase and the second phase is an extended release phase or vice versa. In a further embodiment, the compositions of the present invention providing biphasic release of the drug mycophenolate allow an immediate release of a fraction of the drug into the gastrointestinal tract to provide a rapid onset of action and then a sustained release of the remaining fraction of the drug to provide a prolonged action for an extended duration of time. In a still further embodiment, the rapid release in the first phase induces the immediate onset of the active and the sustained release in the second phase allows the drug level in the blood to be maintained at or below the peak level, but higher than the level obtained with an immediate release dosage form, at the same time after dosing, with the objective of maintaining a suitable and a desirable therapeutic regimen for an extended duration.

In another embodiment, the compositions of the present invention exhibiting biphasic release of the drug mycophenolate allow a sustained release of a fraction of the drug to provide a prolonged action for an extended duration of time followed by an immediate release of a fraction of the drug.

In another embodiment, the compositions of the present invention exhibiting biphasic release of the drug mycophenolate allow a sustained release of a fraction of the drug to provide a prolonged action for an extended duration of time present in a range from about 70% to 99% w/w of mycophenolate as the active agent followed by an immediate release of a fraction of the drug present in a range from about 1% to 30% w/w of mycophenolate as the active agent.

In another embodiment, the compositions of the present invention exhibiting biphasic release of the drug mycophenolate allow an immediate release of a fraction of the drug followed by a sustained release of a fraction of the drug to provide a prolonged action for an extended duration of time.

In another embodiment, the compositions of the present invention exhibiting biphasic release of the drug mycophenolate allow an immediate release of a fraction of the drug present in a range from about 70% to 99% w/w of mycophenolate as the active agent followed by a sustained release of a fraction of the drug to provide a prolonged action for an extended duration of time present in a range from about 1% to 30% w/w of mycophenolate as the active agent.

In an embodiment, small quantities of the drug mycophenolate in a formulation for rapid release can be retained in the formulation and thus may be released at a time after about 30 to about 120 minutes from the initiation of drug release, and are thus included in the prolonged release phase. Similarly, small quantities of the drug incorporated in the prolonged or extended release pharmaceutical entity may be released before about 2 hours, and thus form part of the immediate release phase. According to the present invention, the proportion of the drug contained within the immediate release fraction which is dissolved within about 0.1 to 2 hours is at least about 1% to about 90% by weight of the unit dose composition.

In yet another embodiment, the present invention provides modified release pharmaceutical compositions comprising mycophenolate as the active agent or its pharmaceutically acceptable salts, esters, polymorphs, isomers, prodrugs, solvates, hydrates, or derivatives thereof, at least one release controlling material(s) and optionally one or more pharmaceutically acceptable excipient(s), wherein the said composition exhibits a pH independent biphasic release profile when subjected to in-vitro dissolution and/or upon administration in-vivo.

In another embodiment of the present invention, the pharmaceutical compositions are in the form of pellets or seeds or cores or beads, granules, capsules or tablets/minitablets which comprise an oral modified drug delivery system that: (a) provides a multiphasic release profile of the drug substance that exhibits both immediate and prolonged or sustained release characteristics, (b) constitutes a gradient coating of the drug substance that provides initial first pulse for rapid onset of action and a gradient coating of release controlling material(s) such as those which are hydrophilic or hydrophobic or amphiphilic in nature or mixtures thereof that exhibits prolonged or sustained release in the second phase.

In yet another embodiment of the present invention, modified biphasic release pharmaceutical compositions may comprise of capsules, tablets/minitablets, multilayer tablets/minitablets, multicoated tablets/minitablets. The minitablets are preferably filled into suitable size hard gelatin capsules.

In another embodiment of the present invention, the first phase or immediate release entity may be a pharmaceutical immediate release unit like for example but not limited to an immediate release tablet or pellet, or several such units formulated into a capsule or a tablet; as an immediate release matrix in a tablet; as an immediate release layer, that can be incorporated in a multilayer tablet; as an immediate release coating layer in a multicoated tablet or pellet and the like. In one embodiment of the present invention, the second phase or the prolonged or sustained release entity may be but not limited to a prolonged or sustained release tablet or pellet, or several such units formulated into a capsule or a tablet; as a prolonged or sustained release layer, that can be incorporated in a multilayer tablet; as a prolonged or sustained release core or a prolonged or sustained release coating layer in a multicoated tablet; as prolonged or sustained release pellets within a disintegrating tablet and the like.

In another embodiment, the release controlling polymer(s) of the present invention comprises a polymeric material selected from but not limited to the group comprising pH dependent polymers such as alginates, carbomers, cellulose propionate (lower, medium or higher molecular weight), cellulose acetate propionate, cellulose acetate butyrate, cellulose triacetate or methacrylic acid polymers or a mixture thereof used either alone or in combination thereof; pH independent polymers such as acrylate or methacrylate polymers, or cellulosic polymers; soluble or insoluble polymers; swellable polymers; hydrophilic polymers; hydrophobic polymers; ionic polymers such as calcium carboxymethylcellulose or sodium carboxymethylcellulose; non-ionic polymers such as hydroxypropyl methylcellulose (HPMC K 100CR); synthetic or natural polysaccharide selected from the group comprising alkylcelluloses, hydroxyalkyl celluloses, cellulose ethers, cellulose esters, nitrocelluloses, dextrin, agar, carrageenan, pectin, furcellaran, starch and starch derivatives, and mixtures thereof; cellulosic polymer, methacrylate polymer, carboxyvinyl polymer (Carbopol 71 G), Copolymers of acrylate and methacrylates with quarternary ammonium group (Eudragit®), polyvinylpyrrolidone (PVP), polyvinylpyrrolidone-polyvinylacetate polymer (PVP-PVA) copolymer, ethylcellulose, cellulose acetate, poly(alkyl methacrylate), poly(isodecyl methacrylate), poly(lauryl methacrylate), poly(phenyl methacrylate), poly(alkyl acrylate), poly(octadecyl acrylate), poly(ethylene), poly(alkylene), poly(alkylene oxide), poly(alkylene terephthalate), poly(vinyl isobutyl ether), poly(vinyl acetate), poly(vinyl chloride) and polyurethane or a mixture thereof used either alone or in combination thereof. In a further embodiment, the release controlling polymer(s) of the present invention is gum selected from but not limited to a group comprising xanthan gum, guar gum, gum arabic, carrageenan gum, karaya gum, locust bean gum, acacia gum, tragacanth gum, agar and the like, and mixtures thereof.

In an embodiment of the present invention, dosage forms compositions such as in kits wherein the immediate release entity and the prolonged or sustained release entity are administered simultaneously but separately to give a biphasic or multiphasic release are also encompassed in the present invention. Various formulations are encompassed according to the present invention such as a bilayered tablet comprising an immediate release layer and a sustained release layer or a capsule comprising one or more immediate release tablets and one or more prolonged or sustained release tablets. Immediate release tablets may be prepared by direct compression of mixtures of the drug or salts thereof with diluents, such as microcrystalline cellulose, mannitol, sorbitol, and lactose. Other functional excipients such as disintegrants and lubricants can be added. Choice of these functional excipients as well as diluent is well known to anyone skilled in the art. Alternatively tablets may be prepared by granulation with water of a mixture of the drug or salts thereof with suitable diluent, disintegrant and binding polymer; drying of granulate; blending with a lubricant, followed by compression on a tableting machine. The methods used are those generally described in the pharmaceutical literature.

In another embodiment of the present invention, sustained or prolonged release tablets can be prepared by coating immediate release tablets with a diffusion limiting polymer coating. Suitable polymers can be chosen among ethyl cellulose, methyl methacrylate copolymers such as Eudragit® RS, Eudragit® RL, Eudragit® NE commercialized by Rohm Pharma. Coating methods can consist in spraying a solution of the polymer on the tablets, either in a pan coater or a fluid bed coating apparatus. The solvent may be organic or aqueous, depending on the nature of the polymer used. Alternatively prolonged or sustained release tablets can be prepared by incorporating matrix-forming excipients into the formulation. Such matrix-forming excipients may be hydrophilic polymers, which include hydroxypropyl methylcellulose, hydroxymethylcellulose, hydroxyethylcellulose, and which swell in contact with aqueous liquids, and control release of the drug by diffusion through the swollen polymer network, and are incorporated at a level between 10 and 30% by weight with respect to that of the prolonged release tablet. Otherwise the matrix forming excipient may be a lipid substance, such as hydrogenated castor oil, or carnuba wax, incorporated at a level between 10-40% by weight with respect to the prolonged release tablet.

In another embodiment, the composition can be in the form of a capsule comprising a mixture of prolonged or sustained release pellets and immediate release pellets. The immediate release pellets may be prepared by deposition of the drug suspended in water or an organic solvent or mixtures thereof with a hydrophilic or a lipophilic or amphiphilic material or another suitable polymer to act as a binder, onto a granule. A fluid bed coating apparatus is generally used. Particles may be agglomerated to form spherical granules or pellets, in a high speed mixer granulator, or rotary fluid bed agglomerator. Prolonged or sustained release pellets are prepared by coating immediate release pellets in the same way as described for the tablets. Coating may be carried out, for example, in coating pans or in fluid bed coater-driers. The amount and composition of the coating is adjusted from that used in the tablet, to reduce the permeability of the coating in order to take into account the far greater surface for diffusion in the pellets.

In another embodiment, the composition can be in the form of a tablet comprising a number of prolonged or sustained release coated pellets comprising the drug embedded in a matrix. Alternatively the tablet may consist of a mixture of prolonged or sustained release coated pellets and of immediate release non-coated pellets comprising the drug, embedded in a drug-free matrix. Alternatively the prolonged or sustained release coated pellets may be furthermore coated with a layer comprising the drug and other excipients allowing immediate release from that layer, embedded in a drug-free matrix. The matrix surrounding the pellets should preferably be formulated so that the compression into tablets does not interfere with the integrity of the membrane surrounding the pellets. On contact with fluid the tablet disintegrates, releasing the drug rapidly, from the matrix, or the immediate release pellets, or from the immediate release pellet coating, and then releasing the drug from the prolonged or sustained release pellets slowly.

In another embodiment, the composition can be in the form of a multilayer tablet comprising: (i) one or two prolonged or sustained release layers, comprising the drug and a hydrophilic polymer (preferably a cellulose derivative), (ii) one or more immediate release layers comprising the drug, and possibly, (iii) another layer not comprising the drug, but comprising hydrophilic polymers, such as hydroxypropylcellulose, hydroxyethylcellulose or soluble diluents, such as lactose, sorbitol, mannitol, or hydrophilic polymers and soluble excipients, which layer modulates release of the drug from the prolonged or sustained release layer. Each layer contains other excipients, so as to give suitable properties for compression, lubrication, binding, etc. as is well known to one skilled in art.

In another embodiment, the composition can be in the form of a multicoated tablet comprising: (i) a core comprising the drug and as mycophenolate, optionally with pharmaceutically acceptable excipients, (ii) a polymer coating layer giving slow release of the drug from this core, and (iii) a coating layer comprising the drug which is released rapidly or immediately on contact of the dosage form with fluid. Each portion of the tablet, in particular the inner core, can contain other excipients, so as to give suitable properties for compression, lubrication, and binding as is well known to one skilled in the art. In an embodiment, the composition of the present invention may be in the form of multiparticulates such as pellets, which might be optionally compressed into a tablet for filled into a capsule. Pellets may be prepared by extrusion of wet masses or melts followed by spheronisation.

In a preferred embodiment, the biphasic modified release delivery system of the invention is a two phase system which comprises (1) a first phase in the form of individual granules or particles or beads or core comprising mycophenolate as the active agent or its pharmaceutically acceptable salts, esters, polymorphs, isomers, prodrugs, solvates, hydrates, or derivatives thereof, optionally with pharmaceutically acceptable excipients and (2) a second phase comprising of an outer solid continuous phase in which granules or particles or beads or core of inner solid particulate phase are dispersed and embedded, the outer solid continuous phase which primarily is formed of sustained or prolonged or extended release material formed of one or more hydrophilic or hydrophobic or amphiphilic material/s or mixtures thereof wherein the said composition exhibits a biphasic release profile when subjected to in-vitro dissolution.

The biphasic modified release formulation of the invention is particularly adapted for delivery of mycophenolate and pharmaceutically acceptable salts thereof, without significant initial burst of drug, and wherein release of drug (liberated from the individual dispersed particles forming the inner solid particulate phase) is effectively controlled. Drug upon being released from the particles of the inner phase, in effect, migrates through the outer solid continuous phase and then is released from the formulation into the upper gastrointestinal tract to be available for absorption. As indicated, the inner solid particulate phase will be formed of individual discrete granules or particles or beads or cores each of which contains drug and one or more polymeric materials. In effect, the components of the inner solid particulate phase are in particulate association without having a barrier layer around the individual particles or granules. The outer solid continuous phase is preferably a continuous phase or matrix having the particles or granules including drug (forming the inner solid phase) dispersed throughout and embedded in the continuous outer solid phase.

The pharmaceutically acceptable excipient(s) useful in the composition of the present invention are selected from but not limited to a group of excipients generally known to persons skilled in the art e.g. diluents such as lactose (Pharmatose DCL21), starch, mannitol, sorbitol, dextrose, microcrystalline cellulose, dibasic calcium phosphate, sucrose-based diluents, confectioner's sugar, monobasic calcium sulfate monohydrate, calcium sulfate dihydrate, calcium lactate trihydrate, dextrates, inositol, hydrolyzed cereal solids, amylose, powdered cellulose, calcium carbonate, glycine, and bentonite; disintegrants; binders; fillers; bulking agent; organic acid(s); colorants; stabilizers; preservatives; lubricants; glidants/antiadherants; chelating agents; vehicles; bulking agents; stabilizers; preservatives; hydrophilic polymers; solubility enhancing agents such as glycerin, various grades of polyethylene oxides, transcutol and glycofurol; tonicity adjusting agents; local anesthetics; pH adjusting agents; antioxidants; osmotic agents; chelating agents; viscosifying agents; wetting agents; emulsifying agents; acids; sugar alcohol; reducing sugars; non-reducing sugars and the like used either alone or in combination thereof. The disintegrants used in the present invention include but not limited to starch or its derivatives, partially pregelatinized maize starch (Starch 1500®), croscarmellose sodium, sodium starch glycollate, clays, celluloses, alginates (e.g. Kelton HVCR), pregelatinized corn starch, crospovidone (Kollidon CL-M), gums and the like used either alone or in combination thereof. The lubricants used in the present invention include but not limited to talc, magnesium stearate, calcium stearate, sodium stearate, stearic acid, hydrogenated vegetable oil, glyceryl behenate, glyceryl behapate, waxes, Stearowet, boric acid, sodium benzoate, sodium acetate, sodium chloride, DL-leucine, polyethylene glycols, sodium oleate, sodium lauryl sulfate, magnesium lauryl sulfate and the like used either alone or in combination thereof. The anti-adherents or glidants useful in the present invention are selected from but not limited to a group comprising talc, corn starch, DL-leucine, sodium lauryl sulfate, and magnesium, calcium and sodium stearates, and the like or mixtures thereof. The vehicles suitable for use in the present invention can be selected from but not limited to a group comprising dimethylacetamide, dimethylformamide and dimethylsulphoxide, N-methylpyrrolidone, benzyl benzoate, benzyl alcohol, ethyl oleate, polyoxyethylene glycolated castor oils (commercially available as Cremophor® EL), polyethylene glycol MW 200 to 6000, propylene glycol, hexylene glycols, butylene glycols and glycol derivatives such as polyethylene glycol 660 hydroxystearate (commercially available as Solutrol® HS15). In another embodiment of the present invention, the compositions may additionally comprise an antimicrobial preservative such as Benzyl alcohol preferably at a concentration of 2.0% v/v of the composition: In an embodiment of the present invention, the composition may additionally comprise a conventionally known antioxidant such as ascorbyl palmitate, butylhydroxyanisole, butylhydroxytoluene, propyl gallate and α-tocopherol. In another embodiment, the dosage form of the present invention additionally comprises at least one wetting agent(s) such as a surfactant selected from a group comprising anionic surfactants, cationic surfactants, non-ionic surfactants, zwitterionic surfactants or mixtures thereof. The wetting agents are selected from but not limited to a group comprising oleic acid, glyceryl monostearate, sorbitan monooleate, sorbitan monolaurate, triethanolamine oleate, polyoxyethylene sorbitan monooleate, polyoxyethylene sorbitan monolaurate, sodium oleate, sodium lauryl sulfate and the like or mixtures thereof. In yet another embodiment, the dosage form of the present invention additionally comprises at least one complexing agent such as cyclodextrin selected from a group comprising but not limited to alpha-cyclodextrin, beta-cyclodextrin, betahydroxy-cyclodextrin, gamma-cyclodextrin, and hydroxypropyl beta-cyclodextrin, or the like. In yet another embodiment, the dosage form of the present invention additionally comprises of lipids include but not limited to glyceryl behenate such as Compritol® AT0888, Compritol® HD ATO 5, and the like; hydrogenated vegetable oil such as hydrogenated castor oil e.g. Lubritab®; glyceryl palmitostearate such as Precirol® ATO 5 and the like, or mixtures thereof.

In an embodiment, the compositions of the present invention may be in the coated form. The amount of coating applied is adapted so as to obtain a predetermined dissolution characteristic of the modified release composition. The percentage by weight of the coating on the individual unit dosage form is about 0.01-30% w/w of the composition. The amount of coating applied depends on the predetermined dissolution characteristics of the particular core composition and the desired release profile. However, the amount of coating applied is such that there will be no rupturing problems. The coating may be admixed with various excipients such as plasticizers, anti-adhesives such as, e.g., colloidal silicon dioxide (Aerosil 200), inert fillers, and pigments in a manner known per se. Tackiness of the water-dispersible film-forming substances may be overcome by simply incorporating an anti-adhesive in the coating. The anti-adhesive is preferably a finely divided, substantially insoluble, pharmaceutically acceptable non-wetting powder having anti-adhesive properties in the coating. Examples of anti-adhesives are metallic stearates such as magnesium stearate or calcium stearate, microcrystalline cellulose, or mineral substances such as calcite, substantially water-insoluble calcium phosphates or substantially water-insoluble calcium sulphates, colloidal silica, titanium dioxide, barium sulphates, hydrogenated aluminium silicates, hydrous aluminium potassium silicates and talc.

Examples of plasticizers for use in accordance with the present invention include triacetin, acetylated monoglyceride, rape oil, olive oil, sesame oil, acetyl tributyl citrate, acetyl triethyl citrate, glycerin, sorbitol, diethyloxalate, diethylmalate, diethylmaleate, diethylfumarate, diethylsuccinate, diethylmalonate, dioctylphthalate, dibutylsebacetate, triethylcitrate, tributylcitrate, glyceroltributyrate, polyethyleneglycol, propyleneglycol, 1,2-propyleneglycol, dibutyl sebacate, diethyl sebacate and mixtures thereof. The plasticizer is normally incorporated in an amount of less than about 20% by weight, calculated on the dry matter content of the coating composition.

In an embodiment, the composition of the present invention can be in the form of a tablet-in-tablet which contains a portion of the drug in the form of small tablet. The small tablet is coated with Opadry®/wax/water soluble/insoluble polymer or excipient coating to prevent the diffusion of the water soluble drug from the inner core to outer sustained layer of the polymer drug matrix on exposure to gastric fluids. This small coated tablet is covered by another tablet comprising of remaining portion of the drug. The drug will be released from the small tablet after the outer layer has been completely eroded.

In another embodiment, the present invention encompasses the different approaches used to formulate the compositions. The said approached according to the present invention are as follows:
i) Bilayer tablet which is having an immediate release layer and a sustained release layer both layers comprising mycophenolate, wherein target drug release from the immediate release layer is designed to avoid the lag period which is usually observed in simple/conventional matrix dosage form.
ii) Tablet-in-tablet which is having a portion of drug in the form of small tablet wherein the said small tablet is covered by another tablet with the remaining portion of drug. The drug is released from the small tablet either immediately or in a sustained manner as soon as the outer tablet layer erodes.
iii) Trilayer tablet which is having three layers in which the drug layer has a protection from both side with polymeric material as barrier layer. The drug layer is designed in a manner that drug is released through diffusion mechanism. The barrier layer is designed in such a way that it will be intact through out the drug release and drug will be released through diffusion.
iv) Bilayer tablet having a drug layer and a barrier layer.
v) Timed/programmed release dosage form is prepared by coating of drug loaded pellets using hydrophobic and hydrophilic polymers with different weight build up. Then the coated pellets are compressed with the inert material to get tablets. The tablet composition is designed in such a way that it disintegrates upon exposure to an aqueous environment and then the drug is released from the coated pellets through diffusion mechanism.

In a further embodiment, the present invention provides process of preparing the modified release pharmaceutical compositions according to the present invention comprising mycophenolate or its pharmaceutically acceptable salts, esters, polymorphs, isomers, prodrugs, solvates, hydrates, or derivatives thereof as the active agent.

In a preferred embodiment, the present invention provides process of preparing the modified release pharmaceutical compositions wherein the composition is prepared by a process comprises of the following steps:
i) treating the active agent mycophenolate sodium along with at least one release controlling material(s) which is hydrophilic or hydrophobic or amphiphilic or mixtures thereof,
ii) optionally adding one other active agent(s),
iii) optionally along with one or more pharmaceutically acceptable excipient(s), and
iv) formulating into a suitable dosage form.

In yet another embodiment is provided a method of using the composition of the present invention for the management such as prophylaxis, amelioration and/or treatment of immunosuppressant indicated disease(s)/disorder(s) especially for the treatment and/or prevention of organ, tissue or cellular allograft or xenograft rejection, e.g. after transplant, or the management of immune-mediated diseases (autoimmune diseases), which comprises administering to a subject in need thereof the composition comprising a pharmaceutically effective amount of mycophenolate sodium as the active agent.

In still further embodiment of the present invention is provided use of the pharmaceutical composition comprising pharmaceutically effective amount of mycophenolate sodium in the preparation of a medicament for the management such as prophylaxis, amelioration and/or treatment of immunosuppressant indicated disease(s)/disorder(s) especially for organ, tissue or cellular allograft or xenograft rejection, such as after transplant, or the management of immune-mediated diseases (autoimmune diseases).

The pharmaceutical compositions of the present invention are intended for once-a-day or twice-a-day administration, preferably for once-a-day administration. The once-a-day composition releases the active agent mycophenolate sodium in a desired manner so as to maintain prophylactic and/or therapeutic levels of the active agent in the plasma for modified period of time with reduced gastro-intestinal side effects, and also can be prepared in an easy and cost-effective manner.

The examples of pharmaceutical compositions given below serve to illustrate embodiments of the present invention. However, they do not intend to limit the scope of present invention in any manner whatsoever.

The term ‘q.s.’ wherever appears in the examples is an abbreviation for ‘quantity sufficient’ which is the amount of the excipient in such quantities that is just sufficient for its use in the composition of the present invention.

In an embodiment of the present invention, the composition as mentioned hereinafter in Example-3 was subjected to bioavailability study conducted in healthy adult human subjects. The protocol of the study is as follows:

“A randomized, open label, balanced, three-treatment, three-period, six-sequence, single dose, three-way crossover bioequivalence study of Mycophenolate MR tablet 720 mg of Panacea Biotec Ltd., India under fasting and fed condition with mycophenolate mofetil 1000 mg (Cellcept®500 mg×2) tablets of Roche Laboratories Inc., USA under fasting condition only in 18+6 (stand-by) healthy human adult male subjects.”.

| | Avg MPA Conc. in plasma |
|---|---|
| Time (hrs) | ((μg/ml) (Example-3) |
| 0 | 0.000 |
| 0.33 | 5.202 |
| 0.5 | 7.198 |
| 0.67 | 6.736 |
| 0.833 | 7.106 |
| 1 | 6.619 |
| 1.25 | 5.111 |
| 1.5 | 4.941 |
| 2 | 4.137 |
| 2.5 | 3.761 |
| 3 | 3.030 |
| 3.5 | 2.649 |
| 4 | 2.312 |
| 4.5 | 3.109 |
| 5 | 4.166 |
| 5.5 | 3.740 |
| 6 | 2.816 |
| 6.5 | 2.140 |
| 7 | 1.753 |
| 7.5 | 1.556 |
| 8 | 1.585 |
| 9 | 1.445 |
| 10 | 1.283 |
| 11 | 1.444 |
| 12 | 1.413 |
| 14 | 1.237 |
| 16 | 1.102 |
| 18 | 0.817 |
| 22 | 0.972 |
| 24 | 0.764 |

The results of the ratio of AUC₀₋₁₂ to AUC₁₂₋₂₄ for the composition mentioned as hereinbelow in Example-3 are as follows:

| | AUC₀₋₁₂ | AUC₁₂₋₂₄ | Ratio (AUC₀₋₁₂:AUC₁₂₋₂₄) |
|---|---|---|---|
| Mean | 26.51 | 11.671 | 2.27:1 |

### Example-1

### (80:20) SR:IR

| | | |
|---|---|---|
| S. No. | Ingredient | Quantity/tablet (mg) |

Sustained Release (SR) Layer

| | | |
|---|---|---|
| 1. | Mycophenolate sodium | 630.98 |
| | (equivalent to 576 mg of | |
| | mycophenolic acid) | |
| 2. | Lactose DCL21 | 5.5 |
| 3. | Aerosil 200 | 150.02 |
| 4. | Polyvinyl pyrrolidone | 55 |
| | (PVP K-90) | |
| 5. | Hydroxypropyl methyl | 55 |
| | cellulose | |
| 6. | Polyethylene oxide | 110 |
| | (Polyox WSR 301) | |
| 7. | Polyvinyl pyrrolidone | 27.5 |
| | (PVP K-30) | |
| 8. | Isopropyl alcohol | q.s. |
| | | (lost in processing) |
| 9. | Magnesium stearate | 10.5 |

Immediate Release (IR) Layer

| | | |
|---|---|---|
| 10. | Mycophenolate sodium | 158.3 |
| | (equivalent to 148 mg | |
| | of mycophenolic acid) | |
| 11. | Microcrystalline cellulose | 44 |
| | (Avicel ® PH 101) | |
| 12. | Polyvinyl pyrrolidone | 2.2 |
| | (PVP K-30) | |
| 13. | Isopropyl alcohol | q.s. |
| | | (lost in processing) |
| 14. | Magnesium stearate | 2.2 |

### Procedure:

i) Mycophenolate sodium, lactose anhydrous, colloidal silicon dioxide, polyethylene oxide, hydroxypropyl methyl cellulose and polyvinyl pyrrolidone (PVP K-90) were weighed and passed through #30 s.s. sieve and mixed for 5 mins.
ii) PVP K-30 was dissolved in Isopropyl alcohol and the dispersion was used to granulate the material of step (i). The wet mass was dried and passed through suitable sieves to get desired granules.
iii) Step (ii) granules were lubricated with #40 passed magnesium stearate.
iv) Mycophenolate sodium and microcrystalline cellulose were passed through #30 s.s. sieve and mixed well for 5 mins.
v) PVP K-30 was dissolved in Isopropyl alcohol and the dispersion was used to granulate the material of step (iv). The wet mass was dried and passed through suitable sieves to get desired granules.
vi) The granules of step (v) were lubricated with #40 passed magnesium stearate followed by compression to obtain the tablet.

### Coating Solution Preparation:

vii) Opadry AMB (1 part) was dispersed in water (3 parts) by continuous stirring.
viii) The tablets of step (vi) were coated with the dispersion of step (vii).
ix) The granules of step (iii) were compressed with the coated tablets of step (viii) to obtain tablet in tablet.

### Dissolution Details:

Type of apparatus: USP Type III

### Dips/minute: 15

pH of dissolution medium: 6.8

### Volume: 250 ml

TABLE 1
Cumulative release

| Time (Hrs.) | % released |
|---|---|
| 0 | 0 |
| 1 | 14.8 |
| 2 | 28.5 |
| 4 | 47.4 |
| 6 | 64.2 |
| 8 | 102.7 |

### Example 2

### (80:20) SR:IR

| | | |
|---|---|---|
| S. No. | Ingredient | Quantity/tablet (mg) |

Sustained Release (SR) Layer

| | | |
|---|---|---|
| 1. | Mycophenolate sodium | 630.98 |
| | (equivalent to 576 mg | |
| | mycophenolic acid) | |
| 2. | Lactose DCL21 | 5.5 |
| 3. | Aerosil 200 | 150.02 |
| 4. | Polyvinyl pyrrolidone (PVP K-90) | 55 |
| 5. | Hydroxypropyl methyl cellulose | 55 |
| 6. | Polyethylene oxide (Polyox WSR 301) | 110 |
| 7. | Polyvinyl pyrrolidone (PVP K-30) | 27.5 |
| 8. | Isopropyl alcohol | q.s. |
| | | (lost in processing) |
| 9. | Magnesium stearate | 10.5 |

Immediate Release (IR) Layer

| | | |
|---|---|---|
| 10. | Mycophenolate sodium | 158.3 |
| | (equivalent to 148 mg | |
| | of mycophenolic acid) | |
| 11. | Starch 1500 | 42 |
| 12. | Succinic acid | 22 |
| 13. | Polyvinyl pyrrolidone (PVP K-30) | 2.5 |
| 14. | Isopropyl alcohol | q.s. |
| | | (lost in processing) |
| 15. | Magnesium stearate | 2.5 |

### Procedure:

i) Mycophenolate sodium, lactose anhydrous, colloidal silicon dioxide, polyethylene oxide, hydroxypropyl methyl cellulose, polyvinyl pyrrolidone (PVP K-90) and starch 1500 were weighed and passed through #30 s.s. sieve and mixed for 5 mins.
ii) PVP K-30 was dissolved in Isopropyl alcohol and the dispersion was used to granulate the material of step (i). The wet mass was dried and passed through suitable sieves to get desired granules.
iii) Step (ii) granules were lubricated with #40 passed magnesium stearate.
iv) Mycophenolate sodium, microcrystalline cellulose and succinic acid were passed through #30 s.s. sieve and mixed well for 5 mins.
v) PVP K-30 was dissolved in Isopropyl alcohol and the dispersion was used to granulate the material of step (iv). The wet mass was dried and passed through suitable sieves to get desired granules.
vi) The granules of step (v) were lubricated with #40 passed magnesium stearate followed by compression to obtain the tablet.

### Coating Solution Preparation:

vii) Ethyl cellulose dispersion (1 part) was dispersed in water (4 parts) by continuous stirring.
viii) The tablets of step (vi) were coated with the dispersion of step (vii).
ix) The granules of step (iii) were compressed with the coated tablets of step (viii) to obtain tablet in tablet.

### Dissolution Details:

Type of apparatus: USP Type III

### Dips/minute: 15

pH of dissolution medium: 6.8

### Volume: 250 ml

TABLE 2
Cumulative release

| Time in (Hrs.) | % released |
|---|---|
| 0 | 0 |
| 1 | 19.6 |
| 2 | 31.4 |
| 4 | 51.5 |
| 6 | 68 |
| 7 | 84 |
| 8 | 105.6 |

### Example 3

### (80:20) SR:IR

| | | |
|---|---|---|
| S. No. | Ingredient | Quantity/tablet (mg) |

Sustained Release (SR) Layer

| | | |
|---|---|---|
| 1. | Mycophenolate sodium | 630.98 |
| | (equivalent to 576 mg | |
| | of mycophenolic acid) | |
| 2. | Lactose DCL21 | 22.7 |
| 3. | Aerosil 200 | 5 |
| 4. | Polyvinyl pyrrolidone (PVP K-90) | 25 |
| 5. | Hydroxypropyl methyl cellulose | 150 |
| 6. | Polyethylene oxide (Polyox WSR 301) | 25 |
| 7. | Polyvinyl pyrrolidone (PVP K-30) | 10 |
| 8. | Isopropyl alcohol | q.s. |
| | | (lost in processing) |
| 9. | Magnesium stearate | 10 |
| 10. | Hydroxypropyl methyl cellulose | 55 |
| 11. | Cetostearyl Alcohol | 110 |
| 12. | Magnesium stearate | 10.5 |

Immediate Release (IR) Layer

| | | |
|---|---|---|
| 13. | Mycophenolate sodium | 158 |
| | (equivalent to 148 mg | |
| | of mycophenolic acid) | |
| 14. | Lactose DCL21 | 5.4 |
| 15. | Succinic acid | 20 |
| 16. | Kollidon CLM | 10 |
| 17. | Polyvinyl pyrrolidone (PVP K-30) | 7 |
| 18. | Isopropyl alcohol | q.s. |
| | | (lost in processing) |

### Procedure:

i) Mycophenolate sodium, lactose anhydrous, colloidal silicon dioxide, polyethylene oxide, hydroxypropyl methyl cellulose and polyvinyl pyrrolidone (PVP K-90) were weighed and passed through #30 s.s. sieve and mixed for 5 mins.
ii) PVP K-30 was dissolved in Isopropyl alcohol and the dispersion was used to granulate the material of step (i). The wet mass was dried and passed through suitable sieves to get desired granules.
iii) Step (ii) granules were mixed with hydroxypropyl methyl cellulose and cetostearyl alcohol followed by lubrication with #40 passed magnesium stearate.
iv) Mycophenolate sodium, microcrystalline cellulose, Kollidon CLM and succinic acid were passed through #30 s.s. sieve and mixed well for 5 mins.
v) PVP K-30 was dissolved in Isopropyl alcohol and the dispersion was used to granulate the material of step (iv). The wet mass was dried and passed through suitable sieves to get desired granules.
vi) The granules of step (v) were lubricated with #40passed magnesium stearate followed by compression to obtain the tablet.

### Coating Solution Preparation:

vii) Ethyl cellulose dispersion (1 part) was dispersed in water (4 parts) by continuous stirring.
viii) Opadry White was added to the dispersion of step (vii) & stirred to get a homogeneous dispersion.
ix) The tablets of step (vi) were coated with the dispersion of step (viii).
x) The granules of step (iii) were compressed with the coated tablets of step (ix) to obtain tablet in tablet.

### Dissolution Details:

Type of apparatus: USP Type III

### Dips/minute: 15

pH of dissolution medium: 6.8

### Volume: 250 ml

TABLE 3
Cumulative release

| Time in | 2% coated | 2% coated |
|---|---|---|
| Hrs. | Cumulative | Non Cumulative |
| 0 | 0 | 0 |
| 2 | 24.4 | 24.4 |
| 4 | 40.8 | 16.4 |
| 6 | 60.3 | 19.5 |
| 8 | 87.5 | 27.2 |
| 10 | 97.4 | 9.9 |
| 12 | 102.5 | 5.1 |

### Example 4

### (80:20) SR:IR

| | | |
|---|---|---|
| S. No. | Ingredient | Quantity/tablet (mg) |

Sustained Release (SR) Layer

| | | |
|---|---|---|
| 1. | Mycophenolate sodium | 591 |
| | (equivalent to 540 mg | |
| | of mycophenolic acid) | |
| 2. | Lactose DCL21 | 125.25 |
| 3. | Hydroxypropyl methyl cellulose | 210 |
| 4. | Polyethylene oxide (Polyox WSR 301) | 30 |
| 5. | Polyvinyl pyrrolidone (PVP K-30) | 36 |
| 6. | Isopropyl alcohol | q.s. |
| | | (lost in processing) |
| 7. | Sodium alginate (Kelton HVCR) | 120 |
| 8. | Calcium sulphate | 90 |
| 9. | Magnesium stearate | 12 |

Immediate Release (IR) Layer

| | | |
|---|---|---|
| 10. | Mycophenolate sodium | 158 |
| | (equivalent to 148 mg | |
| | of mycophenolic acid) | |
| 11. | Lactose DCL21 | 5.4 |
| 12. | Succinic acid | 20 |
| 13. | Kollidon CLM | 10 |
| 14. | Polyvinyl pyrrolidone (PVP K-30) | 5 |
| 15. | Isopropyl alcohol | q.s. |
| | | (lost in processing) |
| 16. | Magnesium stearate | 2 |

### Procedure:

i) Mycophenolate sodium, lactose anhydrous, polyethylene oxide and hydroxypropyl methyl cellulose were weighed and passed through #30 s.s. sieve and mixed for 5 mins.
ii) PVP K-30 was dissolved in Isopropyl alcohol and the dispersion was used to granulate the material of step (i). The wet mass was dried and passed through suitable sieves to get desired granules.
iii) Step (ii) granules were mixed with sodium alginate and calcium sulphate followed by lubrication with #40 passed magnesium stearate.
iv) Mycophenolate sodium, microcrystalline cellulose, Kollidon CLM and succinic acid were passed through #30 s.s. sieve and mixed well for 5 mins.
v) PVP K-30 was dissolved in isopropyl alcohol and the dispersion was used to granulate the material of step (iv). The wet mass was dried and passed through suitable sieves to get desired granules.
vi) The granules of step (v) were lubricated with #40passed magnesium stearate followed by compression to obtain the tablet.

### Coating Solution Preparation:

vii) Ethyl cellulose dispersion (1 part) was dispersed in water (4 parts) by continuous stirring.
viii) Opadry White was added to the dispersion of step (vii) & stirred to get a homogeneous dispersion.
ix) The tablets of step (vi) were coated with the dispersion of step (viii).
x) The granules of step (iii) were compressed with the coated tablets of step (ix) to obtain tablet in tablet.

### Dissolution Details:

Type of apparatus: USP Type III

### Dips/minute: 15

pH of dissolution medium: 6.8

### Volume: 250 ml

TABLE 4
Cumulative release

| Time | % age |
|---|---|
| (Hrs.) | release |
| 4 | 33.9 |
| 8 | 62.6 |
| 10 | 75.0 |
| 11 | 86.3 |
| 12 | 90.9 |
| 13 | 101.6 |

### Example 5

### (75:25) IR:SR

| | | |
|---|---|---|
| S. No. | Ingredient | Quantity/tablet (mg) |

Immediate Release (IR) Layer

| | | |
|---|---|---|
| 1. | Mycophenolate sodium | 591 |
| | (equivalent to 540 mg | |
| | of mycophenolic acid) | |
| 2. | Lactose DCL21 | 125.25 |
| 3. | Crospovidone (Kollidon CL-M) | 15 |
| 4. | Polyvinyl pyrrolidone (PVP K-30) | 18.75 |

Sustained Release (SR) Layer

| | | |
|---|---|---|
| 5. | Mycophenolate sodium | 197 |
| | (equivalent to 180 mg | |
| | of mycophenolic acid) | |
| 6. | Lactose DCL21 | 11.2 |
| 7. | Hydroxypropyl methylcellulose | 70 |
| | (HPMC K100 MCR) | |
| 8. | Polyethylene oxide | 26.25 |
| | (Polyox WSR 301) | |
| 9. | Polyvinyl pyrrolidone (PVP K-30) | 7 |
| 10. | Cetostearyl alcohol | 35 |
| 11. | Magnesium stearate | 3.5 |

### Procedure:

i) Mycophenolate sodium, lactose anhydrous and crospovidone were weighed and passed through #40 s.s. sieve and mixed for 5 mins.
ii) PVP K-30 was dissolved in Isopropyl alcohol and the dispersion was used to granulate the material of step (i). The wet mass was dried and passed through suitable sieves to get desired granules.
iii) Step (ii) granules were lubricated with #40 passed magnesium stearate.
iv) Mycophenolate sodium, lactose DCL21, hydroxypropyl methylcellulose, polyethylene oxide and cetosteryl alcohol were weighed & mixed for 5 mins.
v) PVP K-30 was dissolved in Isopropyl alcohol and the dispersion was used to granulate the material of step (iv). The wet mass was dried and passed through suitable sieves to get desired granules.
vi) The granules of step (v) were lubricated with #40passed magnesium stearate followed by compression to obtain the tablet.
vii) The granules of step (iii) were compressed with the tablets of step (vi) to obtain bilayer tablet.

TABLE 5
Cumulative release

| Time | % age |
|---|---|
| (Hrs.) | release |
| 0 | 0 |
| 1 | 85.3 |
| 2 | 89.5 |
| 4 | 98.2 |
| 6 | 103.4 |

### Example 6

### (75:25) SR:IR

| | | |
|---|---|---|
| S. No. | Ingredient | Quantity/tablet (mg) |

Sustained Release (SR) Layer

| | | |
|---|---|---|
| 1. | Mycophenolate sodium | 591 |
| | (equivalent to 540 mg | |
| | of mycophenolic acid) | |
| 2. | Lactose DCL21 | 14 |
| 3. | Sodium alginate (Kelton HVCR) | 50 |
| 4. | Calcium sulphate | 25 |
| 5. | Hydroxypropyl methylcellulose | 200 |
| | (HPMC K 100 MCR) | |
| 6. | Polyethylene oxide (Polyox WSR 301) | 100 |
| 7. | Polyvinyl pyrrolidone (PVP K-30) | 10 |
| 8. | Isopropyl alcohol | q.s. |
| | | (lost in processing) |
| 9. | Magnesium stearate | 10 |

Immediate Release (IR) Layer

| | | |
|---|---|---|
| 10. | Mycophenolate sodium | 197 |
| | (equivalent to 180 mg | |
| | of mycophenolic acid) | |
| 11. | Lactose DCL21 | 5.4 |
| 12. | Crospovidone (Kollidon CLM) | 10 |
| 13. | Polyvinyl pyrrolidone (PVP K-30) | 5 |
| 14. | Isopropyl alcohol | q.s. |
| | | (lost in processing) |
| 15. | Magnesium stearate | 2.5 |

### Procedure:

i) Mycophenolate sodium, lactose anhydrous, sodium alginate, calcium sulphate, polyethylene oxide and hydroxypropyl methyl cellulose were weighed and mixed for 5 mins
ii) PVP K-30 was dissolved in Isopropyl alcohol and the dispersion was used to granulate the material of step (i). The wet mass was dried and passed through suitable sieves to get desired granules.
iii) Step (ii) granules were lubricated with #40 passed magnesium stearate.
iv) Mycophenolate sodium, lactose anhydrous and Kollidon CLM were passed through #40 s.s. sieve and mixed well for 5 mins
v) PVP K-30 was dissolved in isopropyl alcohol and the dispersion was used to granulate the material of step (iv). The wet mass was dried and passed through suitable sieves to get desired granules.
vi) The granules of step (v) were lubricated with #40passed magnesium stearate followed by compression to obtain the tablet.

### Coating Solution Preparation:

vii) Ethyl cellulose dispersion (1 part) was dispersed in water (4 parts) by continuous stirring.
viii) Opadry White was added to the dispersion of step (vii) & stirred to get a homogeneous dispersion.
ix) The tablets of step (vi) were coated with the dispersion of step (viii).
x) The granules of step (iii) were compressed with the coated tablets of step (ix) to obtain tablet in tablet.

TABLE 6
Cumulative release

| Time | % age |
|---|---|
| (Hrs.) | release |
| 0 | 0 |
| 1 | 15.3 |
| 2 | 24.3 |
| 4 | 41.3 |
| 6 | 54 |
| 8 | 60 |
| 10 | 101.3 |

### Example 7

### (80:20) IR:SR

| | | |
|---|---|---|
| S. No. | Ingredient | Quantity/tablet (mg) |

Immediate Release (IR) Layer

| | | |
|---|---|---|
| 1. | Mycophenolate sodium | 630 |
| | (equivalent to 575 mg | |
| | of mycophenolic acid) | |
| 2. | Starch 1500 | 119.25 |
| 3. | Crospovidone (Kollidon CL-M) | 24 |
| 4. | Polyvinyl pyrrolidone (PVP K-30) | 18.75 |
| 5. | Magnesium stearate | 8 |

Sustained Release (SR) Layer

| | | |
|---|---|---|
| 6. | Mycophenolate sodium | 158 |
| | (equivalent to 148 mg | |
| | of mycophenolic acid) | |
| 7. | Lactose DCL21 | 22.26 |
| 8. | Hydroxypropyl methylcellulose | 70 |
| | (HPMC K100 MCR) | |
| 9. | Polyvinyl pyrrolidone (PVP K-30) | 8.75 |
| 10. | Ethyl cellulose | 87.5 |
| 11. | Magnesium stearate | 3.5 |

### Procedure:

i) Mycophenolate sodium, starch 1500 and crospovidone were weighed and passed through #40 s.s. sieve and mixed for 5 mins.
ii) PVP K-30 was dissolved in Isopropyl alcohol and the dispersion was used to granulate the material of step (i). The wet mass was dried and passed through suitable sieves to get desired granules.
iii) Step (ii) granules were lubricated with #40 passed magnesium stearate.
iv) Mycophenolate sodium, lactose DCL21 and hydroxypropyl methylcellulose were weighed & mixed for 5 mins.
v) PVP K-30 was dissolved in Isopropyl alcohol and the dispersion was used to granulate the material of step (iv). The wet mass was dried and passed through suitable sieves to get desired granules.
vi) The granules of step (v) were mixed with ethyl cellulose and lubricated with #40passed magnesium stearate followed by compression to obtain the tablet.
vii) The granules of step (iii) were compressed with the tablets of step (vi) to obtain bilayer tablet.

TABLE 7
Cumulative release

| Time | % age |
|---|---|
| (Hrs.) | release |
| 0 | 0 |
| 1 | 89.3 |
| 2 | 94.6 |
| 4 | 99.2 |
| 6 | 104.6 |

1. Modified release pharmaceutical composition comprising mycophenolate as an active agent or a pharmaceutically acceptable salt, ester, polymorph, isomer, prodrug, solvate, hydrate, or derivative there of, at least one release controlling material and optionally one or more pharmaceutically acceptable excipient, wherein the composition exhibits a multiphasic release profile when subjected to in-vitro dissolution and/or upon administration in-vivo and wherein the composition releases the drug in a manner so that drug levels are maintained above the therapeutically effective concentration (EC) constantly for an extended duration of time.

2. The composition according to claim 1, wherein the composition provides a biphasic/multiphasic release of mycophenolate such that Area-under-the-drug plasma concentration-time-curve up to 12 hours (AUC₀₋₁₂) is relatively close to the Area-under-the-drug plasma concentration-time-curve from 12-24 hours (AUC₁₂₋₂₄) thereby ensuring that the therapeutic concentrations of the active agent are maintained for a longer duration of time.

3. The composition according to claim 2, wherein the ratio of AUC₀₋₁₂ to AUC₁₂₋₂₄ is from about 4:1 to about 1:1 thereby ensuring that the therapeutic concentration of the active agent is maintained for a longer duration of time.

4. The composition according to claim 3, wherein the ratio of AUC₀₋₁₂ to AUC₁₂₋₂₄ is from about 3:1 to about 1:1 thereby ensuring that the therapeutic concentration of the active agent is maintained for a longer duration of time.

5. The composition according to claim 3, wherein the ratio of AUC₀₋₁₂ to AUC₁₂₋₂₄ is from about 2.5:1 to about 1:1 thereby ensuring that the therapeutic concentration of the active agent is maintained for a longer duration of time.

6. The composition according to claim 1, wherein the composition can release the drug in a manner such that the difference between the maximum plasma concentration of the drug (Cmax) and the minimum plasma concentration of the drug (Cmin), and in turn the flux as herein defined ((Cmax−Cmin)/Cavg) is relatively less and provides a flattened drug release profile for an extended time period wherein the Area-under-curve (AUC) practically remains unchanged for a substantially longer period of time.

7. The composition according to claim 1, wherein the active agent is adapted to release over a predetermined time period exhibiting a biphasic release profile, wherein a first phase is an immediate release phase and a second phase is an extended release phase or the first phase is an extended release phase and the second phase is an immediate release phase.

8. The composition according to claim 1, wherein the composition provides for biphasic release of the drug mycophenolate allowing an immediate release of a fraction of the drug into the gastrointestinal tract to provide a rapid onset of action and then a sustained release of the remaining fraction of the drug to provide a prolonged action for an extended duration of time.

9. The composition according to claim 1, wherein a rapid release in a first phase induces immediate onset of the active agent and sustained release in a second phase allows the drug level in the blood to be maintained at or below the peak level, but higher than the level obtained with an immediate release dosage form, at the same time after dosing, with the objective of maintaining a suitable and a desirable therapeutic regimen for an extended duration.

10. The composition according to claim 1, wherein a sustained release of a fraction of the drug provides a prolonged action for an extended duration of time along with an immediate release of a fraction of the drug in between after the onset of the sustained release of the drug in order to maintain therapeutically effective concentration of the drug.

11. The composition according to claim 1, wherein the composition exhibits biphasic release of the drug mycophenolate allowing a sustained release of a fraction of the drug to provide a prolonged action for an extended duration of time followed by an immediate release of a fraction of the drug.

12. The composition according to claim 11, wherein the composition exhibiting biphasic release of the drug mycophenolate allows a sustained release of a fraction of the drug to provide a prolonged action for an extended duration of time present in a range from about 70% to 99% w/w of mycophenolate as the active agent followed by an immediate release of a fraction of the drug present in a range from about 1% to 30% w/w of mycophenolate as the active agent.

13. The composition according to claim 1, wherein the composition exhibiting biphasic release of the drug mycophenolate allows an immediate release of a fraction of the drug followed by a sustained release of a fraction of the drug to provide a prolonged action for an extended duration of time.

14. The composition according to claim 13, wherein the composition exhibiting biphasic release of the drug mycophenolate allows an immediate release of a fraction of the drug present in a range from about 70% to 99% w/w of mycophenolate as the active agent followed by a sustained release of a fraction of the drug to provide a prolonged action for an extended duration of time present in a range from about 1% to 30% w/w of mycophenolate as the active agent.

15. The composition according to claim 1, wherein the composition exhibits a pH independent biphasic release profile when subjected to in-vitro dissolution and/or upon administration in-vivo.

16. The composition according to claim 1, in the form of a pellet seed core bead, granule, capsule or tablet/minitablet which comprises an oral modified drug delivery system that: (a) provides a multiphasic release profile of the drug substance that exhibits both immediate and prolonged or sustained release characteristics, (b) constitutes a gradient coating of the drug substance that provides initial first pulse for rapid onset of action and a gradient coating of release controlling material(s).

17. The composition according to claim 1, wherein the release controlling material(s) are hydrophilic hydrophobic or amphiphilic in nature or a mixture thereof that exhibits prolonged or sustained release in the second phase.

18. The compositions according to claim 1, in a form of a capsule, tablet/minitablet, multilayer tablet/minitablet or multicoated tablet/minitablet.

19. The composition according to claim 1, wherein the release controlling material comprises a polymeric material selected from the group comprising pH dependent polymers, pH independent polymers, gums, lipid agents and mixtures thereof.

20. The composition according to claim 19, wherein the pH dependent polymer is selected from a group comprising alginates, carbomers, cellulose propionate (lower, medium or higher molecular weight), cellulose acetate propionate, cellulose acetate butyrate, cellulose triacetate or methacrylic acid polymers or a mixture thereof used either alone or in combination thereof.

21. The composition according to claim 19, wherein the pH independent polymer is selected from a group comprising acrylate or methacrylate polymers, or cellulosic polymers; soluble or insoluble polymers; swellable polymers; hydrophilic polymers; hydrophobic polymers; ionic polymers such as calcium carboxymethylcellulose or sodium carboxymethylcellulose; non-ionic polymers such as hydroxypropyl methylcellulose; synthetic or natural polysaccharide selected from the group comprising alkylcelluloses, hydroxyalkyl celluloses, cellulose ethers, cellulose esters, nitrocelluloses, dextrin, agar, carrageenan, pectin, furcellaran, starch and starch derivatives, and mixtures thereof cellulosic polymer, methacrylate polymer, Copolymers of acrylate and methacrylates with quarternary ammonium group (Eudragit®), polyvinylpyrrolidone (PVP), polyvinylpyrrolidone-polyvinylacetate polymer (PVP-PVA) copolymer, ethylcellulose, cellulose acetate, poly(alkyl methacrylate), poly(isodecyl methacrylate), poly(lauryl methacrylate), poly(phenyl methacrylate), poly(alkyl acrylate), poly(octadecyl acrylate), poly(ethylene), poly(alkylene), poly(alkylene oxide), poly(alkylene terephthalate), polyvinyl isobutyl ether), poly(vinyl acetate), poly(vinyl chloride) and polyurethane or a mixture thereof used either alone or in combination thereof.

22. The composition according to claim 19, wherein the gum is selected from a group comprising xanthan gum, guar gum, gum arabic, carrageenan gum, karaya gum, locust bean gum, acacia gum, tragacanth gum, agar and the like or mixtures thereof.

23. The composition according to claim 19, wherein the lipid agent is selected from a group comprising glyceryl behenate such as Compritol® AT0888, Compritol® HD ATO 5, and the like; hydrogenated vegetable oil such as hydrogenated castor oil e.g. Lubritab®; glyceryl palmitostearate such as Precirol® ATO 5 and the like, or mixtures thereof.

24. The composition according to claim 1, which may be administered as a kit wherein the immediate release entity and the prolonged or sustained release entity are administered simultaneously but separately to give a biphasic or multiphasic release.

25. The composition according to claim 24, wherein sustained or prolonged release tablet can be prepared by coating immediate release tablet with a diffusion limiting polymer coating.

26. The composition according to claim 1, in the form of a capsule comprising a mixture of prolonged or sustained release pellets and immediate release pellets.

27. The composition according to claim 1 in the form of a tablet comprising a number of prolonged or sustained release coated pellets comprising the drug embedded in a matrix.

28. The composition according to claim 1, wherein the composition can be in the form of a multilayer tablet comprising: (i) one or two prolonged or sustained release layers, comprising the drug and a hydrophilic polymer (preferably a cellulose derivative), (ii) one or more immediate release layers comprising the drug, and possibly, (iii) another layer not comprising the drug, but comprising hydrophilic polymers, such as hydroxypropylcellulose, hydroxyethylcellulose or soluble diluents, such as lactose, sorbitol, mannitol, or hydrophilic polymers and soluble excipients, which layer modulates release of the drug from the prolonged or sustained release layer.

29. The composition according to claim 1, wherein the composition can be in the form of a multicoated tablet comprising: (i) a core comprising the drug and as mycophenolate, optionally with pharmaceutically acceptable excipients, (ii) a polymer coating layer giving slow release of the drug from this core, and (iii) a coating layer comprising the drug which is released rapidly or immediately on contact of the dosage form with fluid.

30. The composition according to claim 1, wherein the biphasic modified release delivery system is a two phase system which comprises (1) a first phase in the form of individual granules or particles or beads or core comprising mycophenolate as the active agent or its pharmaceutically acceptable salts, esters, polymorphs, isomers, prodrugs, solvates, hydrates, or derivatives thereof, optionally with pharmaceutically acceptable excipients and (2) a second phase comprising of an outer solid continuous phase in which granules, particles, beads or core of inner solid particulate phase are dispersed and embedded, the outer solid continuous phase which primarily is formed of sustained or prolonged or extended release material formed of one or more hydrophilic or hydrophobic or amphiphilic material/s or mixtures thereof wherein the composition exhibits a biphasic release profile when subjected to in-vitro dissolution.

31. The composition according to claim 41, wherein the excipient(s) are selected from a group comprising diluents; disintegrants; binders; fillers; bulking agents; organic acid(s); colorants; stabilizers; preservatives; lubricants; glidants/antiadherants; chelating agents; vehicles; stabilizers; preservatives; hydrophilic polymers; solubility enhancing agents; tonicity adjusting agents; pH adjusting agents; antioxidants; osmotic agents; chelating agents; viscosifying agents; wetting agents; emulsifying agents; acids; sugar alcohols; reducing sugars; non-reducing sugars and the like used either alone or in combination thereof.

32. The composition according to claim 1, wherein the composition further comprises an antioxidant, at least one wetting agent(s), at least one complexing agent, lipids and the like, or a mixture thereof.

33. The composition according to claim 1, wherein the composition is in a coated form.

34. The composition according to claim 1 in the form of a tablet-in-tablet which contains a portion of the drug in the form of small tablet.

35. The composition according to claim 1, wherein the composition is prepared by a process comprising the steps of: i) treating the active agent mycophenolate sodium along with at least one release controlling material(s) which is hydrophilic or hydrophobic or mixtures thereof, ii) optionally adding one other active agent(s), iii) optionally adding one or more pharmaceutically acceptable excipient(s), and iv) formulating into a suitable dosage form.

36. The method of using the composition according to claim 1 for the management such as prophylaxis, amelioration and/or treatment of immunosuppressant indicated disease(s)/disorder(s) especially for the treatment and/or prevention of organ, tissue or cellular allograft or xenograft rejection, or the management of immune-mediated diseases (autoimmune diseases), which comprises administering to a subject in need thereof the composition comprising a pharmaceutically effective amount of mycophenolate sodium as the active agent.

37. (canceled)

38. (canceled)

39. The pharmaceutical composition according to claim 1 intended for once-a-day or twice-a-day administration.

40. (canceled)

41. The composition according to claim 28, wherein each layer further comprises an excipient.
